(19) United States
(12) Patent Application Publication
(10) Pub. No.: US 2005/0171436 A1
(43) Pub. Date: Aug. 4, 2005

(54) Raman spectroscopy for monitoring drug-eluting medical devices

(76) Inventors: Richard H. Clarke, Boston, MA (US); M. Edward Womble, Watertown, MA (US)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 10/754,158

(22) Filed: Jan. 9, 2004

(51) Int. Cl. A61B 6/00; A61B 5/05

(52) U.S. Cl. 600/476; 600/407; 600/473; 600/478

(57) ABSTRACT

The present invention provides low-resolution Raman spectroscopic systems and methods for in situ monitoring of drug-eluting devices in a lumen of a subject. A preferred system can employ multi-mode radiation in making in situ Raman spectroscopic measurements of the lumen and/or device. For example, a system can include a light source such as a multi-mode laser, and a light detector to measure spectral patterns and differentiates spectral features of drugs released in a target region. Drug-release curves can be extrapolated or otherwise predicted using the Raman spectrum taken during or subsequent to device insertion and/or activation.

### BACKGROUND OF THE INVENTION

The technical field of this invention is Raman spectroscopy and, in particular, the use of Raman scattering to monitor in situ drug-eluting medical devices, for example, drug-eluting stents used for vascular repair.

Coronary heart disease is a major cause of death and disability, accounting for substantial health costs. Underlying most cases is development of atherosclerotic lesions in coronary arteries, or at least, coronary artery narrowing generally due to plaque. Initially, balloon angioplasty was used to enlarge narrowing arteries in a preventative strike against heart disease. Such procedures successfully opened narrowed arteries in most patients and relieved symptoms such as chest pain. Over months, however, recurrent chest pain developed in many patients as restenosis, or a “re-narrowing” of the arteries, occurred at the treatment site.

Coronary stents offered improvements when used in conjunction with balloon angioplasty, but also had drawbacks due to scar tissue formation at the treatment site. Stents are generally metallic mesh devices placed at the treatment site in the artery to provide support to the artery wall, and in general, can result in a larger flow channel. Although stents significantly decrease restenosis, unfortunately, scar formation can form at the treatment site. For example, in approximately 20% to 30% of patients, scar tissue grows through openings of the stent, narrowing the flow channel therethrough and causing, in many ways, the same issues associated with restenosis.

Drug-eluting coronary stents, however, can reduce scar tissue formation thus improving treatment outcome. Scar tissue formation can be reduced or eliminated by various antiproliferate drugs, such as Sirolimus (Rapamune™ American Home Products Corp.). The drug is combined with a polymer that is applied to an outer aspect of the stent as a thin coating. The stent is inserted into a vessel, and the coating activated to begin release of the drug and consequently, drug absorption by vessel walls in proximity to the stent. Various studies show drug-eluting stents dramatically decrease chances of detrimental scar tissue growth. For example, positive results are described in M. Morice et al., N. Engl. J. Med., 346, 1773 (2002); P. W. Serruys et al., Circulation, 106 798 (2002); and F. Listro et al., Circulation 105, 1883 (2002).

Unfortunately, performance of a drug-eluting stent can only be determined by repeated patient evaluations over time in an attempt to identify signs of restenosis or other detrimental changes in a subject patient. Generally, it is unknown if the drug-polymer coating is correctly eluting a drug in sufficient amounts for substantially full therapeutic benefits. The amount of drug eluted can be different than expected because of, for example, “pre-elution” of the drug occurring while the device is in its packaging during shipping and/or storage, elution occurring after removal of the device from its packaging but before insertion into a lumen, and insertion and activation of the polymer coating in a lumen of the subject.

Thus, there is a need for monitoring of drug-eluting medical devices.

### SUMMARY OF THE INVENTION

The present invention is directed to low-resolution Raman spectroscopic systems for monitoring of drug-eluting medical devices before and/or after insertion and activation in a lumen of a subject. The system can include a light source such as a multi-mode laser, a light collector and/or a light dispersion element, and a detector to measure spectral patterns that indicate the presence of the drug released from the medical device. Based on a spectral response of a target (e.g., the lumen wall), the presence, or absence, of the drug can be determined, and an amount of drug that will be eluted in a lumen of a subject can be predicted.

In one aspect of the invention, an optical sensor system is employed in making Raman spectroscopic measurements of a drug-eluting device, its packaging container, and or the device after insertion and activation in a lumen of a subject to determine the presence or absence of a drug. Systems according to the invention can also allow in situ Raman spectroscopic measurements of a lumen wall adjacent or in close proximity to an inserted and activated drug-eluting device.

Accordingly, in one aspect, the present invention provides a system for detecting the presence or absence of a drug using low-resolution Raman spectroscopy in a target region and can allow for a prediction of an amount of drug that will be eluted in the lumen of the subject over a time period. The target region can be a device, its packaging container and/or the device in a lumen of a subject. The system can include a catheter comprising an excitation fiber through which multi-mode radiation can propagate to irradiate the target region. A multi-mode laser, such as a GaAs laser diode, can produce the multi-mode radiation. A low-resolution dispersion element can receive scattered radiation, e.g., that light scattered by the target, and separate the received radiation into different wavelength components. A detection array optically coupled to the dispersion element or other light collecting element can detect least some of those wavelength components. A processor receives data from the detection array and processes that data to determine the presence or absence of the drug, and can lead to a prediction of drug-release curves of the device corresponding a time period.

In use, the multi-mode laser irradiates the target to produce a Raman spectrum composed of scattered electromagnetic radiation characterized by a particular distribution of wavelengths. The Raman spectrum results from scattering of the laser radiation as it interacts with the target.

A collector element collects and communicates the scattered radiation from the target to the dispersion element. Thus, the collector element can be an optical fiber with a first end positioned for collecting scattered radiation, and a second end positioned in proximity to the dispersion element. One or more filters can be employed, e.g., notch filters, to reduce or attenuate optical noise, for example, excitation source background noise.

The dispersion element distributes (e.g., separates) the scattered radiation into different wavelength components. This can be accomplished by a diffraction grating, for example. At least a portion of the wavelength components are detected by the detection array which can be a charged-coupled diode (CCD) array. The resolving power of the dispersion element determines the position of specific wavelengths in the detection array in such way that a signal from a particular diode in the array will generally correspond to the same or similar narrow range of wavelengths.

The processor receives and processes the signals and/or other data from the detection array. For example, the processor can store data corresponding to background noise of the medical device in an unactivated state prior to insertion into the subject. After insertion and activation of that (or a similar) device in the subject, the processor can receive data from the detection array corresponding to measurements taken in the lumen of the subject, and separate the background noise attributable to the medical devices itself. The remaining Raman spectrum then corresponds to an amount of drug released from the medical device. In another feature of the invention, the processor can predict a drug-release curve for a time period longer that the actual in situ Raman sampling time interval. Thus, based on a relatively short time interval, a drug-release curve can be extrapolated or otherwise predicted for a significantly longer time period.

In another aspect, the invention provides methods for detecting the presence or absence of a drug released from a drug-eluting medical device inserted and activated in a lumen of a subject. The method includes providing a catheter generally paralleling one as described herein. Background Raman features of the medical device before installation and activation are known or can be determined via, for example, Raman spectral analysis. After installation and activation of the device, Raman features, taken in situ, can be used to verify and measure the rate of drug elution from the medical device by monitoring the appearance and intensity of the Raman signals from the drug as it is released. The background features can be differentiated from the in situ features, thus enabling a determination of the amount of drug released and/or elution rates.

Systems according to the present invention can be suitable for measuring drug levels in the sub-milligram range. In a further related aspect, systems such as those described herein can predict drug release curves for extended periods, e.g., 90-days, based on an amount of drug released from the medical device over a relatively shorter period, e.g., during the stenting procedure.

### BRIEF DESCRIPTION OF THE DRAWINGS

The invention will be more fully understood from the following detailed description taken in conjunction with the accompanying drawings, in which:

FIG. 1 is a block diagram of a sensor system suitable for use with the invention;

FIG. 2 is a schematic, partially cut-away perspective view of an apparatus for spectral analysis;

FIG. 2A is a cross section view of the apparatus of FIG. 2 taken along section line 1A-1A;

FIG. 3 is a partially cross sectional view of an alternative apparatus for spectroscopic analysis according to the invention;

FIG. 4 is a further partially cross sectional view of an alternative apparatus for spectroscopic analysis according to the invention; and

FIG. 5 is a Raman spectrum of a scar-tissue growth-inhibiting drug;

### DETAILED DESCRIPTION OF THE INVENTION

The invention is directed to in situ monitoring of drug-eluting medical devices such as stents inserted into a lumen of a subject (e.g., a blood vessel), using low-resolution Raman spectroscopy to monitor the extent and/or rate of a drug released prior to, during, and/or after stenting of an atherosclerotic lesion, for example. Thus, evaluation of a packaged and/or an inserted and activated stent is performed to determine drug-release characteristics that can be expected from that stent, and to verify adequate release of the drug at a time when the stent can be easily replaced. Although the invention is described in terms of stents, it will be obvious to one skilled in the art that the invention can be used with other drug-eluting devices, and in other fields, such as for detection of other blood-borne drugs and/or components within a vessel or body cavity, or detection of other drugs absorbed by a lumen wall such as a wall of a blood vessel.

General background information on Raman spectral analysis can be found in U.S. Pat. No. 5,139,334, issued to Clarke and incorporated herein by reference, which teaches a low resolution Raman analysis system for determining certain properties related to hydrocarbon content of fluids. The system utilizes a Raman spectroscopic measurement of the hydrocarbon bands and relates specific band patterns to the property of interest. See also, U.S. Pat. No. 6,208,887 also issued to Clarke and incorporated herein by reference, which teaches a low-resolution Raman spectral analysis system for determining properties related to in vivo detection of samples based on a change in the Raman scattered radiation produced in the presence or absence of a lesion in a lumen of a subject.

The present invention provides a Raman system for monitoring drug-eluting devices before insertion into a patient or after the device has been inserted and activated in a lumen of a subject based on the difference in the Raman spectrum patterns associated with components of the eluted drug. In one application, the present invention can be used, specifically, as a quality control measure to test packaged devices.

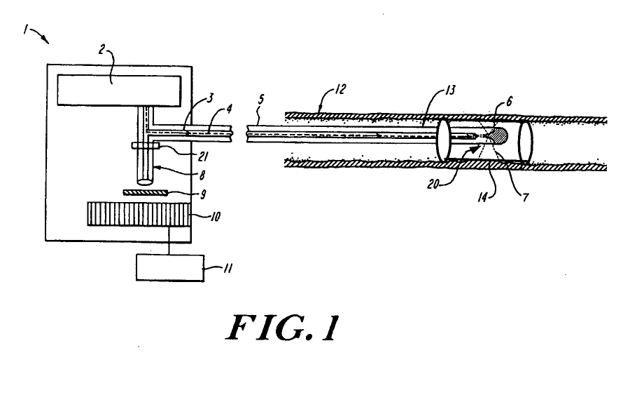

FIG. 1 is a block diagram of a drug-eluting stent 14 inserted in a lumen 12 of a subject and a low-resolution Raman spectroscopy system 1 according to the invention for monitoring release of a drug from the stent. System 1 has a multi-mode laser source 2 connected to an excitation fiber 3, that carries multi-mode laser radiation 4 between a first end of a catheter 5 and a second end of the catheter disposed near a light directing element 6 which directs the laser radiation in a outward direction producing directed radiation 7. The laser radiation exits the catheter 5 via an opening 20 and irradiates a target. The laser radiation scatters in accord with a Raman scattering and is received by a collection bundle 8 through which the radiation travels to a low-resolution dispersion element 9 that serves to disperse the scattered light into different wavelength components that are detected by a detection array 10 and analyzed by a processor 11.

The excitation fiber 3 is connected at a first end to the multi-mode laser 2 and has a second end adjacent to the light directing element 6. Multi-mode laser radiation 4 is carried through the excitation fiber 3, exiting at the second end towards the light directing element 6 which directs the radiation in a sideways direction. Preferably, at least a second portion of the excitation fiber is disposed within a catheter 13 sized to be slidably received by a vessel or other lumen in proximity to the inserted stent 14 or other drug-eluting device. The directed radiation 7 exits the catheter 13 through an opening 20 and irritates a portion of a target such as the inserted stent 14 or lumen wall in proximity to the stent. The opening 20 can be a radial opening having a lens or radiation-transparent covering around the catheter 13, or an orifice either with or without a lens, for example. The light directing element 6 can be for example, conical or flat in shape depending on the size and shape of the opening 20 in the catheter. The light directing element 6 can be a material that is reflective, refractive or diffusive. Where the stent 14 is of a mesh design, the directed radiation 7 can be focused through the mesh of the stent. Raman scattered radiation from the target is collected by the collection bundle 8, which may optionally have a notched filter 21 to remove noise components. The scattered radiation is dispersed into various components by the dispersion element 9 and detected to the detection array 10, which is preferably, a charged-coupled device (CCD) array using diodes.

The resolving power of the dispersion element 9 determines the position of specific wavelength components in the detection array 10 in such a way that the signal from a particular diode in the array will typically correspond to the same (or a similar) narrow range of wavelengths. A low-resolution dispersion element can provide greater transmission of scattered radiation to the detector array. For example, a low-resolution diffraction grating with wider slits than a typical diffraction grating can be used, providing greater transmission of incident scattered radiation to the detector array. Thus, the combination of a low cost, high energy multi-mode laser and a low loss dispersion element provides an inexpensive low-resolution Raman spectroscopy system that can provide a high intensity signal.

The processor 11 selects a particular diode (or diodes) of the array 10 according to the property, e.g., the drug components, to be measured and receives signals corresponding to the diodes illuminated by wavelength components from the dispersion element 9. Signals received from multiple diodes relating to multiple wavelength components can be arithmetically divided to form intensity ratios. The processor 11 can compare these ratios with known values or a correlating function to obtain an estimate of the chemical constituent or property of interest. In a preferred embodiment, the processor can correct received signals for background scatter caused by devices or other characteristics in the target area. For example, background scatter caused by the drug-eluting device can be compensated for to determine a Raman spectrum for the drug eluted from the device absent that background scatter.

By way of background, it will be understood that multi-mode laser radiation energy encountering a target region can be distributed in several distinct modes: absorption, reflection and scattering. Scattering can occur either where the distributed radiation wavelength is unchanged from the incoming wavelength (e.g., Raleigh Scattering), or alternatively, where the distributed wavelengths are altered from that of the incoming wavelengths (e.g., Raman Scattering). Scattering will occur when a target is irradiated with a beam of monochromatic light of frequency w; preferably selected so that it is not strongly absorbed by the target. The resulting electromagnetic field induces a polarizability change in target molecules, and this interaction results in a transfer of energy between the molecules in the target and an electromagnetic wave, as described in Ferraro et al., Introductory Raman Spectroscopy, Academic Press, San Diego, 1994.

A time variance of the electric field, E₀ cos wt, of the radiation passing a molecule will distort its electronic structure and produce an induced dipole in the direction of the electric field. If the polarizability, α, is introduced as the proportionality constant between the electric field and the induced dipole moment, then the induced dipole can be expressed as:
μ_{ind}=αE₀ cos wt. [1]

For a vibrating molecule that is not spherically symmetric, the polarizability along a direction can vary about an average value expressed according to the relationship:
α=αₐᵥ+Δα cos w_{vib}t. [2]

The induced dipole will vary with time according to the relationship:
μ_{ind}=[αₐᵥ+Δα cos w_{vib}t][E₀ cos wt]. [3]

Thus, using the trigonometric relation:
2 cos m cos n=cos(m+n) [4]
Eq. 3 is equivalent to:
μ_{ind}=αₐᵥE₀ cos wt+(Δα)E₀[cos(w+w_{vib})t+cos(w−w_{vib})t]. [5]

The first term of Eq. 5 corresponds to radiation that is scattered without any change in the frequency, w, of the light, and is identified as Raleigh scattering. The second term of Eq. 5 describes an energy-exchange interaction that depends on the non-spherical, or anisotropic, part of the polarizability and involves frequencies shifted from that of the incident radiation by an amount that depends on the vibrational frequency of the molecules in the target. Thus, the second term is Raman scattering, with the frequency of the light, w, changed by an amount±w_{vib}, equal to a molecular vibration. The vibrational frequencies observed are specific to a given molecular structure, and the chemical makeup of the sample can be determined by the characteristic vibrational frequencies observed.

It is through use of those so-called “fingerprint” vibrational frequencies, unique to each particular species in the target, that allow monitoring of the released drug components against a background of other chemical signature vibrations that constitute the stented site within the artery wall.

Thus, since a Raman measurement is the difference in wavelength between the returned scattered light and the laser radiation excitation line, an excitation line that has a larger spectral full width at half-maximum causes a proportional loss of resolution in the resulting Raman measurement. However, this reduction of resolution is generally offset by the advantages of lower cost and increased signal intensity. The increased signal intensity is a result of a higher energy laser source and wider slits in the diffraction grating allowing more light into the detector array. Since the spectrometer system resolution has been reduced by the use of a multi-mode laser, for example, the width of the slits can be increased with negligible effect on the overall resolution. Additionally, a charged-coupled device detector array can be matched to the lower resolution laser source and the wider dispersion element by reducing the number of elements (e.g., diodes) in the array. For example, instead of a 4,096 element diode array, a system can implement a 2,048 element diode array without significantly affecting the overall resolution of the system.

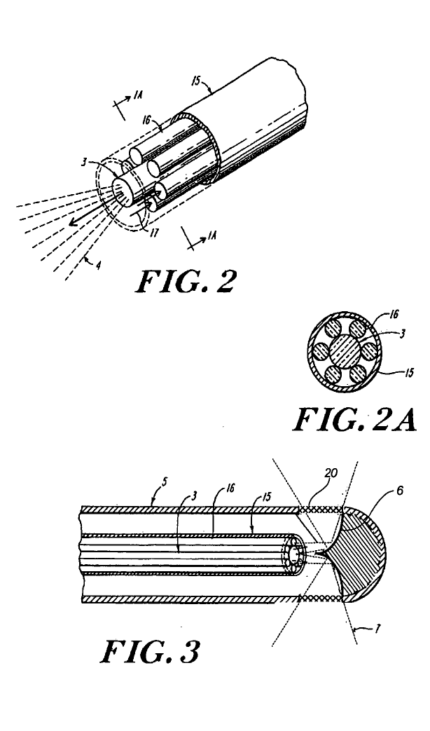

FIG. 2 shows one embodiment of the invention for spectroscopic analysis that includes a casing or sheath 15, and an excitation fiber 3 through which radiation can be propagated and emitted as a conical pattern of excitation radiation 4. The apparatus further includes a number of fibers 16, which receive Raman scattered radiation 17 from the surrounding lumen such as a vessel wall in proximity to a drug-eluting medical device. Although illustrated as optical fibers, it will be apparent that means can be any light waveguide or assembly of optical elements known in the art for collection of radiation from the lumen.

FIG. 2A is a cross sectional view along the sectional line 1A-1A of the apparatus shown in FIG. 2, illustrating the relative positions of the excitation fiber 3 and the collection fibers 16, as well as the protective sheath 15.

FIG. 3 is another apparatus for spectroscopic analysis according to the invention, which includes a catheter 5 that has an excitation fiber 3 and collection fibers 16, surrounded by a sheath 15. The catheter 5 also includes a distal, conical, light-directing element 6 which directs an annular beam of laser radiation in a sideways direction through a ring-like opening or window 20 to produce directed light 7 used to irradiate a portion of the drug eluting device or vessel in proximity to the eluting device. In a preferred embodiment, the catheter 5 is flexible and adapted to be introduced into a lumen of a subject in proximity where a drug-eluting stent has been inserted and activated to release a drug. The catheter 5 can be combined with, for example, an angioplasty catheter such that one catheter can perform both functions, e.g., balloon angioplasty and Raman spectroscopy.

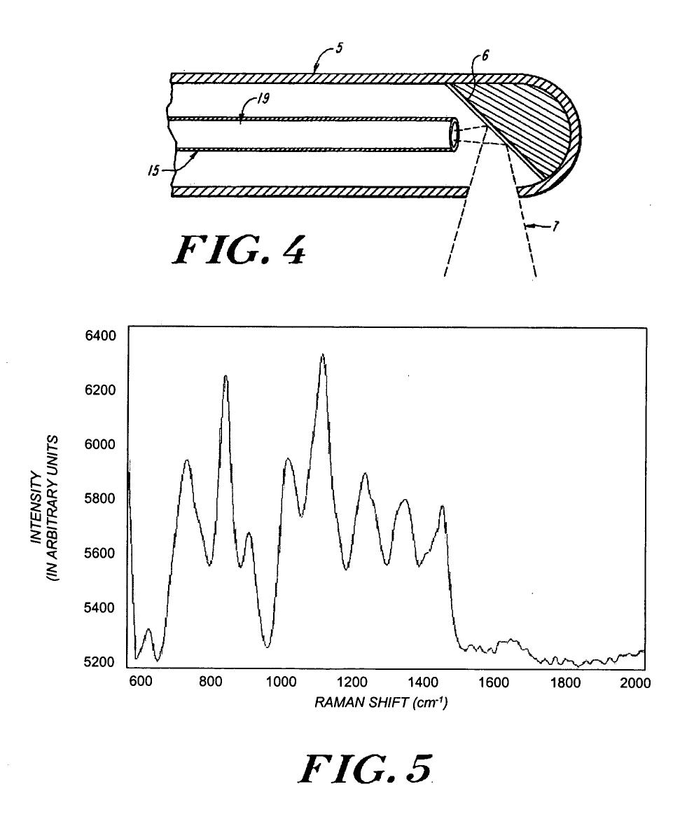

FIG. 4 is an alternative apparatus which includes a single fiber 19 surrounded by a sheath 15 in a catheter 5. The fiber 19 serves as both an excitation fiber and a collection fiber. The fiber 19 directs multi-mode laser radiation to a light-directing element 6 which directs the laser radiation in a sideways direction to irradiate a portion of the drug eluting device or vessel in proximity to the drug-eluting device.

Advances in the field of solid-state lasers have introduced several important laser sources into Raman analysis. For high-resolution Raman systems, the laser linewidth must be severely controlled, often adding to the cost of the excitation source and the system as a whole. For low-resolution Raman spectroscopy, however, the strategy of relinquishing resolution details in favor of emphasizing essential identifying spectral features, allows the use of a low cost, light energy multi-mode laser which can be used with a low-resolution system, according to a preferred embodiment of the present invention, is available in higher power ranges (e.g., between 50 milliwatts (mw) and 1,500 mw) than is available with a traditional single mode laser (generally less than 150 mw). The higher power of a multi-mode laser increases the amount of scattered radiation available to the spectrometer system. The sensitivity of the low-resolution system increases at least linearly with the laser power.

Raman spectra can be obtained at around typical room temperatures using, for example, a R-2001™ fiberoptic-based spectrometer system, commercially available from Raman Systems, Inc., although systems can also be used. In particular, however, the system preferably uses a laser source with a wavelength of between approximately 300 nm and approximately 1,500 nm, and more preferably with a wavelength of between approximately 600 nm and 1,000 nm, and even more preferably at approximately 785 nm at a power level of between approximately 50 milliwatts and 300 milliwatts, more preferably approximately 150 milliwatts measured at the target. A 785 nm laser (or one having a wavelength of approximately 785 nm) source can reduce fluorescence interference while collecting Raman spectra from targets and minimize target heating. The laser preferably generates a light having a line width of between about between 1 nm and about 10 nm, and preferably having a line width of at least about 2 nm. Low-resolution spectra can be taken over a range of approximately 100 cm⁻¹ to approximately 5,000 cm⁻¹, and preferably over a range of approximately 400 cm⁻¹ to approximately 3,000 cm⁻¹, at a resolution of approximately 1 cm⁻¹ to 40 cm⁻¹, more preferably on the order of approximately 10 cm⁻¹ to 30 cm⁻¹, and still more preferably of approximately 15 cm⁻¹, thus providing a wide vibrational range suitable for many drug-eluting device monitoring applications. It will be appreciated that the wavelength, power and range of the Raman system can vary depending on the characteristics of the drug-eluting device, as well as the characteristics of the drug to be detected.

Typically, a drug-eluting medical device can release a drug over a period exceeding hours, days, and weeks or even months. The device can begin eluting the drug shortly after manufacture and packaging, for example, and continue eluting while in storage. It is possible, therefore, that insufficient drug amounts remain in the stent coating to effect an optimal therapeutic benefit to a patient. Thus, in a preferred embodiment, Raman spectrums are acquired from the device and/or package to determine an amount of drug previously eluted prior to inserting the stent into a lumen of a subject. Alternatively, or in addition, the drug-eluting stent can again be irradiated using Raman spectroscopy just prior to insertion into a lumen of a subject. This can ensure adequate drug reserves in the drug-containing coating before beginning the insertion procedure.

It will be appreciated that a drug-eluting medical device, when either packaged and stored, or when inserted and activated, can cause background noise when low-resolution Raman scattering takes place, and it is advantageous to differentiate or otherwise remove from a Raman scattering any wavelength components attributable to any un-released drug components held by the medical device. Thus, a preferred method comprises determining a background scattering of a drug-eluting stent before insertion and activation in a patient to determine a Raman scattering attributable to the device. The background scattering can then be differentiated or otherwise removed from in situ Raman scattering resulting in a determination of the drug released from the medical device.

During insertion of the drug-eluting device in a lumen, the drug-containing coating can be activated to release the drug in therapeutic quantities via, for example, applying UV radiation to the coating. To validate proper activation, and sufficient drug elution, Raman spectroscopy can again be utilized to detect the presence or absence of the drug. In a preferred embodiment, the stent or a wall of the lumen in proximity to the stent is irradiated and resulting Raman spectrums are analyzed to determine the quantity of drug released, if any.

It will be appreciated, however, that continuous monitoring of the stent over the entire drug-eluting period (e.g., 9-months) is difficult or even impossible. Thus, in a preferred embodiment, a drug-eluting stent is monitored for a period shorter than its entire drug-eluting life span, and the results from that shorter period are used to predict, via extrapolation for example, the drug-eluting characteristics expected for the longer drug-eluting life span. For example, Raman spectrums can be obtained shortly after insertion and again multiple times thereafter for a time period, e.g., minutes and/or hours. These Raman spectrums can be analyzed to predict drug-release curves predicting an amount of drug that will be released over a longer time period, e.g., hours and/or months.

This provides in situ analysis of an inserted and activated stent at a time when correction of an improperly activated or otherwise defective stent is possible without waiting for a subject's recurrent symptoms.

Thus, methods for monitoring a drug-eluting medical device according to a preferred embodiment of the invention include providing for a low-resolution Raman spectroscopy device such as one described herein for directing laser radiation at a target region to determine the presence or absence of a drug. For example, the amount of drug present in a drug-eluting device can be measured using low-resolution Raman spectroscopy before and after insertion in a lumen of a subject. The returned Raman spectrums can be analyzed to predict the amount of drug that will be eluted by the device over a time period. Multiple spectroscopy samples can be taken over a time period of seconds or minutes, for example, to determine a rate of release of a drug. These samples are processed to remove background scattering noise attributable to, among other things, the medical stent and drug-containing polymer coating, and can be processed, for example via a partial least-squares analysis, to predict an expected drug-release curve for the device over its drug-eluting life-span.

In a preferred embodiment, a drug-eluting device in situ is irradiated with radiation suitable for inducing Raman scattering as noted above. Scattered radiation is collected from a target region, generally proximal to the drug-eluting device. A Raman spectrum is determined using the collected radiation, which is then analyzed to determine the presence or absence of at least one drug in the target region. A drug-release curve can be predicted using a rate of drug-release, and such can be accomplished by taking several Raman spectrum measurement over time. This may be done in situ either in the original device packaging or at the time of the procedure.

As a demonstration that the approach described above is suitable achieving the objectives of the invention, a low-resolution Raman spectrum of the CYPHER™ Sirolimus (Rapamune™)—Eluting Coronary Stent manufactured by Cordis Corporation was obtained to determine that the drug can be detected via low-resolution Raman spectroscopy. A Raman spectrum of a 1-milligram (mg) Sirolimus tablet was taken in a 10 second scan using an RSL-1 model portable fiberoptic-based low-resolution Raman system. FIG. 5 is the spectrum obtained and displays rich spectral detail, with peaks characteristic of phenyl rings, amide and carbonyl substituents, thus identifying the chemical makeup of Sirolimus. It will be appreciated that other drugs, and specifically, other drugs suitable for release over time, can also be detected by these methods and apparatus disclosed. In FIG. 5, the horizontal axis shows the Raman shift in cm⁻¹, and the vertical axis is in arbitrary scattering intensity units.

With knowledge that Raman spectroscopy can identify the drug Sirolimus, measurement of the background Raman features of the drug-coated stent itself is necessary. As noted above, the stent is coated with a drug-containing polymer that can be, e.g., UV activated and covalently bonded to a surface of the metal stent. Encapsulated in the polymeric coating is the drug agent at a total concentration generally on the order of micrograms, which is released slowly by diffusion from its polymeric matrix over time. To properly evaluate the release of the drug agent, detection of the drug at the microgram level is preferably, and differentiation of the released drug spectral features from background sources of low-resolution Raman scattering signals, such as those arising from the stent itself as well as the organic polymer coating, is also preferable.

The initial Raman spectra of a drug-eluting stent surface generally reveals how much of the signature bands from Sirolimus within the polymer matrix are detectable at the microgram levels when presented against the polymeric background peaks over the same spectral region as had been observed in the preliminary results shown in FIG. 5. The preliminary spectrum obtained on the drug itself at the milligram level suggest sufficient signal-to-noise to detect the drug at microgram levels, and the ability to distinguish the drug—including background spectral interference—from stent coatings.

With use of the background spectral features of the combined drug stent surface, release of the active Sirolimus agent from the inserted and activated stent is possible. In a preferred operation, the drug is released from the stent surface to the artery wall over a period of approximately 90 days, although maintaining uniform release over that period is difficult. Even so, Raman monitoring is possible prior to, and during the stenting procedure, with a full time-dependent curve of drug release obtained by measuring an initial growth of the Raman peaks from the releasing drug over a short initial monitoring period, e.g., several minutes, and using extrapolated results from that initial monitoring data.

One skilled in the art will appreciate further features and advantages of the invention based on the above described embodiments. Accordingly, the invention is not to be limited by what has been particularly shown and described, except as indicated by the appended claims. All publication and references cited herein are expressly incorporated herein by reference in their entirety.

1. A system for monitoring a drug-eluting device in using low-resolution Raman spectroscopy comprising:
a catheter having a first end and a second end with an excitation fiber extending therebetween, the excitation fiber suitable to transmit multi-mode radiation from the first end to the second end to irradiate a target region;
a multi-mode laser coupled to the first end of the excitation fiber, the laser generates multi-mode radiation for irradiating the target region to produce a Raman spectrum consisting of scattered electromagnetic radiation;
a low-resolution dispersion element positioned to receive and separate the scattered radiation into different wavelength components;
a detection array, optically aligned with the dispersion element for detecting at least some of the wavelength components of the scattered light; and
a processor for processing the data from the detector array to monitor a drug eluted from the medical device.

2. The system of claim 1, wherein the target region is any of the group consisting of a device package, a device, and a lumen in a subject.

3. The system of claim 1, wherein the catheter further comprises:
a light directing element optically coupled to the second end of the excitation fiber to direct the laser radiation from the excitation fiber to the target region.

4. The system of claim 3, wherein the light directing element directs the laser radiation out a side of the catheter.

5. The system of claim 1, wherein the system has a resolution of between approximately 1 cm⁻¹ and approximately 40 cm⁻¹.

6. The system of claim 5, wherein the system has a resolution of approximately 15 cm⁻¹.

7. The system of claim 1, wherein the multi-mode laser produces a laser light with a wavelength of approximately 785 nanometers.

8. The system of claim 7, wherein the laser is a GaAs laser diode.

9. The system of claim 1, wherein the multi-mode laser produces a laser light with a power of between approximately 50 milliwatts and 1,500 milliwatts measured at the target.

10. The system of claim 9, wherein the multi-mode laser produces a laser light with a power of approximately 150 milliwatts measured at the target.

11. The system of claim 1, wherein the multi-mode laser produces a laser light with a line width of between approximately 1 nm and 10 nm.

12. The system of claim 11, wherein the multi-mode laser produces a laser light with a line width of at least 2 nm.

13. The system of claim 1, wherein the detection array detects a spectral range between approximately 400 cm⁻¹ and approximately 3,000 cm⁻¹.

14. The system of claim 1, wherein the wavelength components are separated by a resolution ranging from about 10 cm⁻¹ to about 100 cm⁻¹.

15. A method for detecting a drug-release curve indicating presence of a drug released from a drug-eluting device using low-resolution Raman spectroscopy comprising:
determining a Raman spectrum for a background of the drug-eluting device;
determining a Raman spectrum for a target in proximity of the drug-eluting device;
processing the target spectrum and the background spectrum to isolate the target spectrum from the background spectrum;
predicting a drug-release curve over a time period based on the processed spectrums.

16. The method of claim 16, wherein the step of determining a Raman spectrum for a target in proximity of the drug-eluting device comprises determining a Raman spectrum for any of the group consisting of device package, a device, and a lumen in a subject.

17. The system of claim 15, wherein the multi-mode laser produces a laser light with a power of between approximately 50 milliwatts and 1,500 milliwatts measured at the target.

18. The method of claim 17, wherein the multi-mode laser produces a laser light with a power of approximately 150 milliwatts measured at the target.

19. The method of claim 15, wherein the multi-mode laser produces a laser light with a line width of between approximately 1 nm and 10 nm.

20. The method of claim 19, wherein the multi-mode laser produces a laser light with a line width of at least 2 nm.

21. The method of claim 15, wherein the detection array detects a spectral range between approximately 400 cm⁻¹ and approximately 3,000 cm⁻¹.

22. The method of claim 15, wherein the wavelength components are separated by a resolution ranging from about 10 cm⁻¹ to about 100 cm⁻¹.

23. The method of claim 15, further comprising:
providing a catheter comprising an excitation fiber through which multi-mode radiation can propagate, the excitation fiber having a first end optically coupled to a multi-mode laser, and a second end positioned in optical alignment with a light directing element to direct radiation to a target within the lumen;
inserting the catheter in proximity to the target;
activating the multi-mode laser to irradiate the target to produce the target spectrum consisting of scattered electromagnetic radiation;
collecting a portion of the scattered radiation;
separating the collected radiation into different wavelength components using a low-resolution dispersion element;
detecting at least some of the wavelength components of the scattered light using a detection array; and
processing the data from the detection array to detect the presence of the drug released by the drug-eluting device.

24. The method of claim 15, further comprising identifying the components of the target from the data.

25. The method of claim 15, wherein the step determining a Raman spectrum for a target comprises inserting a catheter into a lumen of a subject.

26. The method of claim 25, wherein the lumen is a blood vessel.

27. The method of claim 15, wherein the step of determining a Raman spectrum for drug-absorbing tissue comprises detection of a drug released by the drug-eluting medical device.

28. The method of claim 27, wherein the drug is a scar tissue inhibitor.

29. The method of claim 15, wherein the step of predicting drug-release over a time period further comprises applying a partial least squares analysis to extract chemometric information from the data.

30. A method for determining the presence or absence of a drug using Raman scattered radiation comprising:
irradiating a target region with radiation suitable for inducing Raman scattering;
collecting Raman scattered radiation from the target region;
determining a Raman spectrum from the collected radiation; and
analyzing the Raman spectrum to determine the presence or absence of at least one drug in the target region.

31. The method of claim 30, wherein the step of irradiating a target region comprises irradiating any of the group consisting of a drug-eluting device, a drug-eluting device package, and a lumen of a subject.

32. The method of claim 30, wherein the step of irradiating a device further comprises providing multi-mode laser radiation.

33. The method of claim 32, wherein the laser radiation has a wavelength of between approximately 300 nm and approximately 1,500 nm.

34. The method of claim 32, wherein the laser radiation has a power of between approximately 50 mw and approximately 1,500 mw measured at the target.

35. The method of claim 32, wherein the laser radiation has a line width of between approximately 1 nm and approximately 10 nm.

36. The method of claim 30, wherein the step of determining a Raman spectrum further comprises separating the collected radiation into one or more wavelength components.

37. The method of claim 37, wherein the wavelength components are separated by a resolution ranging from about 10 cm⁻¹ and about 100 cm⁻¹.

38. The method of claim 30, wherein the step of determining a Raman spectrum further comprises determining a spectral range of between about 400 cm⁻¹ and about 3,000 cm⁻¹.

39. The method of claim 30, further comprising:
providing a catheter comprising an excitation fiber through which multi-mode radiation can propagate, the excitation fiber having a first end optically coupled to a multi-mode laser, and a second end positioned in optical alignment with a light directing element to direct radiation to a target;
positioning the second end of the catheter in proximity to the target;
activating the multi-mode laser to irradiate the target;
collecting a portion of the scattered radiation;
separating the collected radiation into different wavelength components using a low-resolution dispersion element;
detecting at least some of the wavelength components of the scattered light using a detection array; and
processing the data from the detection array to detect the presence of the drug released by the drug-eluting device.

40. The method of claim 39, wherein the step of positioning the second end of the catheter further comprises inserting the second end of the catheter into a lumen of a subject.

41. The method of claim 40, wherein the lumen is a blood vessel.

42. The method of claim 30, wherein the step of analyzing the Raman spectrum further comprises differentiating background noise from the Raman spectrum.

43. The method of claim 42, wherein the background noise comprises a Raman scattering of the drug-eluting device.

44. The method of claim 30, further comprising predicting a drug-release curve based on the analyzed Raman spectrum.

45. The method of claim 44, wherein the drug-release curve is over a time period greater than the time period of the collected Raman scattered radiation.
